(19) United States
(12) Patent Application Publication
(10) Pub. No.: US 2017/0027958 A1
(43) Pub. Date: Feb. 2, 2017

(54) FULVESTRANT COMPOSITIONS

(71) Applicants: THEMIS MEDICARE LIMITED, Mumbai (IN); NEVAKAR, LLC, Bridgewater, NJ (US)

(72) Inventors: Dinesh Shantilal PATEL, MUMBAI (IN); SACHIN DINESH PATEL, MUMBAI (IN); SHASHIKANT PRABHUDAS KURANI, MUMBAI (IN); MADHAVLAL GOVINDLAL PATEL, MUMBAI (IN); Kumaresh Soppimath, Skillman, NJ (US)

(73) Assignees: THEMIS MEDICARE LIMITED, MUMBAI (IN); NEVAKAR, LLC, Bridgewater, NJ (US)

(21) Appl. No.: 15/292,810

(22) Filed: Oct. 13, 2016

Related U.S. Application Data
(63) Continuation-in-part of application No. 14/242,973, filed on Apr. 2, 2014.

(30) Foreign Application Priority Data
Apr. 2, 2013 (IN) 1287/MUM/2013

(51) Int. Cl. A61K 31/565; A61K 47/10; A61K 47/44; A61K 9/00

(52) U.S. Cl. CPC A61K 31/565; A61K 9/0019; A61K 47/10; A61K 47/44

(57) ABSTRACT

The inventive subject matter provides ready to inject fulvestrant compositions with improved solubility and stability, and methods for preparing the same. Contemplated compositions include fulvestrant at a concentration of greater than 100 mg/ml, and maintain degradation of the fulvestrant at a level of less than 5 wt % when stored over at least three months at 25° C.

This application is a Continuation-in-part of U.S. patent application Ser. No. 14/242,973 filed on 2 Apr. 2014 and claims priority from India Application Number 1287/MUM/2013 dated 2 Apr. 2013. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is fulvestrant compositions.

### BACKGROUND

The background description includes information that may be useful in understanding the present invention. It is not an admission that any of the information provided herein is prior art or relevant to the presently claimed invention, or that any publication specifically or implicitly referenced is prior art.

Breast cancer is the most common cancer amongst women in many countries, affecting approximately one in eight women during their lives. The risk of breast cancer increases as women age, and the aging population is set to give rise to an increase in its prevalence, especially amongst postmenopausal women.

Fulvestrant is a drug treatment of hormone receptor-positive metastatic breast cancer in postmenopausal women with disease progression following anti-estrogen therapy. Fulvestrant is an estrogen antagonist that competitively binds to and down-regulates estrogen receptors in human breast cancer cells. It inhibits the growth of tamoxifen-resistant and estrogen-sensitive breast cancer cells.

The chemical name of Fulvestrant is 7-alpha-[9-[(4,4,5,5,5-pentafluoropentyl)sulfinyl]nonyl]estra-1,3,5(10)-triene-3,17-beta-diol. The molecular formula is C₃₂H₄₇F₅O₃S and its structural formula is shown by formula I:

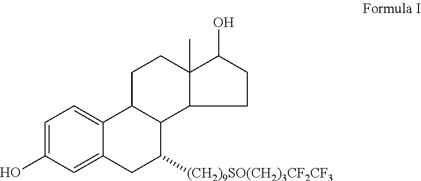

The currently marketed product provided by Faslodex® is a clear, colorless to yellow, viscous solution for injection containing 50 mg/ml fulvestrant. The inactive ingredients of the currently approved product include high concentrations of benzyl alcohol and benzyl benzoate as co-solvents, and castor oil as a release rate modifier. It is supplied in sterile single pre-filled 5 ml syringes for intramuscular injection, and multiple syringes may be required per month depending on the recommended dose and dosing schedule. The composition must be refrigerated at 2-8° C., and should be brought to room temperature before administration.

Unfortunately, due to the poor solubility of fulvestrant in the Faslodex solvent system, a large volume must be injected to the patient in order to receive the full dose, often requiring multiple injections. Additionally, Faslodex is associated with injection site pain, nausea, vomiting and loss of appetite, with a likely cause being the presence of a substantial volume of ricinoleic acid containing castor oil.

Some efforts have been made to provide Faslodex formulations that can be administered at lower volumes, or that reduce some of the side effects associated with the administration.

For example, international patent application number PCT/IN2013/000235 to Palepu teaches storage stable fulvestrant-containing compositions including a DMSO solvent, oil mixtures that are free of castor oils and castor oil derivatives, and a benzyl benzoate or benzyl alcohol sustained release member. Preferred volume ratios of solvent:oil:sustained release member include 1.3:1:1.7 and 1:1:1. Unfortunately, while Palepu focuses on the elimination of castor oil to avoid side effects associated therewith (e.g., gastrointestinal disturbances), Palepu apparently fails to appreciate that larger volumes of benzyl benzoate and benzyl alcohol are often associated with pain at injection sites. Additionally, many of Palepu's formulations were apparently unable to achieve substantial solubility increases when compared to Faslodex (i.e., >100 mg/ml).

Other efforts have been made to provide fulvestrant compositions that are less viscous and more transparent, thereby being useful for rapid onset of action. For example, U.S. patent application publication number 2014/0296191 to Patel et al. teaches fulvestrant compositions utilizing diethylene glycol monoethyl ether as the sole solvent. Unfortunately, Patel only reports a solubility of 58.80 mg/ml for its fulvestrant formulations, which yet again requires large injection volumes.

Thus, there is still a need for improved Fulvestrant compositions with increased solubility and stability for fulvestrant.

### SUMMARY OF THE INVENTION

Applicant surprisingly discovered that the solubility and stability of fulvestrant in diethylene glycol monoethyl ether (DEGEE) can be greatly improved by providing a small concentration (e.g., ≦5 volume per volume (v/v) %) of a co-solvent such as benzyl alcohol. In contrast, when a larger concentration (e.g., >5 v/v %) of the benzyl alcohol was included, the solubility significantly decreased, especially where castor oil was not present.

The inventive subject matter provides ready to inject fulvestrant compositions with improved solubility and stability, which can remain clear and colorless for a period of at least 180 days. Contemplated compositions include fulvestrant at a concentration of greater than 100 mg/ml, and maintain degradation of the fulvestrant at a level of less than 5 weight (wt) % when stored over at least three months at 25° C.

DEGEE can be included in the compositions as a primary solvent or solubilizer, for example, in concentrations of at least 10 v/v %, at least 20 v/v %, at least 30 v/v %, at least 40 v/v %, at least 50 v/v %, at least 60 v/v %, at least 70 v/v %, at least 80 v/v %, at least 85 v/v % of the composition. Viewed from a different perspective, DEGEE can be included in concentrations of at least 10 v/v %, at least 20 v/v %, at least 30 v/v %, at least 40 v/v %, at least 50 v/v %, at least 60 v/v %, at least 70 v/v %, at least 80 v/v %, at least 90 v/v %, or even at least 95 v/v % of the solvent system used to dissolve fulvestrant. Additionally or alternatively, one or more alkyl derivatives of DEGEE could be included as a primary solvent, including for example diethylene glycol monomethyl ether, diethylene glycol mono-iso-propyl ether, diethylene glycol mono-n-propyl ether, diethylene glycol mono-n-butyl ether, diethylene glycol mono-iso-butyl ether, or diethylene glycol mono-n-hexyl ether.

DEGEE is a well studied solvent, which has been tested for its safety and toxicity, and has been reported to be safe for therapeutic use through various routes of administration. DEGEE advantageously has a viscosity and density that makes it easily flowable and syringeable, making it easy to withdraw and administer to patients. Where DEGEE is present in high concentrations in fulvestrant formulations provided herein, the fulvestrant formulations can advantageous have a viscosity and density that makes the formulation easily flowable and syringeable. Additionally, DEGEE has several health advantages over known excipients used in preparing fulvestrant compositions, including glycofurol, which is thought to be a tissue irritant that is both hepatotoxic and nephrotoxic, cremophor EL, which is believed to cause anaphylactic shocks due to its tendency to trigger histamine production when injected, and castor oil, which when administered parenterally in large volumes has been reported to cause widespread disruption of cell processes as a result of the action of ricin, a type 2 ribosome-inactivating protein.

Still further, DEGEE can enhance the absorption of fulvestrant in mammals when injected intramuscularly, and can thus offer an improved pharmacological effect for the intended purpose. In the formulations presented herein, it is preferable to use DEGEE having a purity of at least 99%, more preferably at least 99.7% or at least 99.9%.

A first co-solvent can comprise between 1-10 v/v % of the composition (or solvent system), more preferably between 1-7 v/v %, and even more preferably between 1-5 v/v % or between 1-4 v/v % of the composition (or solvent system). Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. Contemplated co-solvents include, among other things, benzyl alcohol, ethanol, other pharmaceutically acceptable alcohols, dimethyl sulfoxide, glycofurol, N-methyl pyrrolidone, propylene glycol, polyethylene glycols, Solketal, glycerol formal, and acetone. Wherein present in such low concentrations, it is contemplated that the co-solvent will not cause or contribute to toxicity, or substantial pain or inflammation at the injection site.

Optionally, at least one of a release rate modifier and a second co-solvent can be included in some contemplated high solubility fulvestrant compositions without significantly affecting the overall solubility. The release rate modifier(s) can modify the rate of release of the fulvestrant from the drug delivery system, and can include an oil, a castor oil, a medium chain triglycerides (MCT) oil, a fractionated oil, triglycerides, diglycerides, monoglycerides, medium chain fatty acid triglycerides, caprylic/capric triglycerides, oleoyl polyoxy-6 glycerides, behenates, propylene glycol fatty acid diesters (e.g., glyceroltrilaurate, glyceroltrimyristate, glyceroltripalmitate and glyceroltristearate), or any other suitable modifiers. Moreover, biodegradable release rate modifiers such as poly (ε-caprolactone) (PCL), poly (lactide acid) (PLA), polyglycolides (PGA), polyglyconate, polyanhydrides, polyorthoesters, polydioxanone, polyalkylcyanoacrylates and poly (lactic-co-glycolic acid) (PLGA)-based release modifiers can be present. It should be appreciated that one or multiple release rate modifiers can be present in contemplated compositions, and that one or multiple co-solvents for DEGEE can be present. Each release rate modifier and co-solvent can be included in any suitable concentration, including between 1-5 w/v %, between 1-5 v/v %, between 1-10 w/v %, between 1-10 v/v %, between 1-15 w/v %, between 1-15 v/v %, between 1-20 v/v %, between 1-25 v/v %, between 1-35 v/v %, between 1-45 v/v %, between 1-55 v/v %, between 1-65 v/v %, between 1-75 v/v %, less than 60 v/v %, less than 50 v/v %, less than 40 v/v %/o, less than 25 v/v %, less than 15 v/v %, less than 10 v/v %, less than 5 v/v %, or even less than 3 v/v % of the fulvestrant composition or the fulvestrant solvent system.

It is an object of the inventive subject matter to provide a formulation that can deliver therapeutically effective amounts of fulvestrant in minimal volumes to thereby decrease pain and increase patient compliance and ease of use. Therefore, in some aspects, the fulvestrant can be present in the ready to inject composition in a concentration of at least 110 mg/ml, at least 125 mg/ml, at least 150 mg/ml, at least 175 mg/ml, or even at least 200 mg/ml or higher. Viewed from a different perspective, the fulvestrant can be present in the ready to inject composition in a concentration between 100-300 mg/ml, between 110-300 mg/ml, between 110-250 mg/ml, between 125-275 mg/ml, or between 150-250 mg/ml.

In some other aspects, the fulvestrant composition can be formulated to maintain degradation of the fulvestrant at a level of less than 5 wt %, more preferably less than 2 wt %, and more preferably less than 1 wt % or less than 0.5 wt % when stored over at least three months at 25° C. (e.g., at least four months, at least five months, at least six months). Additionally or alternatively, the fulvestrant composition can be formulated to maintain degradation of the fulvestrant at a level of less than 5 wt %, more preferably less than 2 wt %, and more preferably less than 1 wt % or less than 0.5 wt % when stored over at least three months at between 2-8° C. (e.g., at least four months, at least five months, at least six months). Additionally or alternatively, the fulvestrant composition can be formulated to maintain degradation of the fulvestrant at a level of less than 5 wt %, more preferably less than 2 wt %, and more preferably less than 1 wt % or less than 0.5 wt % when stored over at least three months at 40° C. (e.g., at least four months, at least five months, at least six months).

Thus, the inventors also contemplate a container (e.g., a vial, an ampoule, an intravenous bag, a syringe, a cartridge) that may be configured as single-use or multi-use containers, and methods of manufacturing ready to inject fulvestrant composition containing articles. Where the container is configured as a multi-use container, the container includes a quantity of the fulvestrant composition that is suitable for independent and multiple administrations (e.g., 2, 3, 4, 5 administrations). Viewed from a different perspective, methods of suppressing formation of a plurality of degradation products of fulvestrant in solution are contemplated. The fulvestrant compositions can be formulated to remain clear and colorless when stored for a period of at least 30 days, or even at least 180 days at a temperature of between 2-40° C., inclusive, even in the presence of Oxygen in the head space of the containers.

The inventive subject matter also provides for use of a fulvestrant composition as described herein for the treatment or prevention of a disease, for example, a cancer, a physiological disease or a pathological disease.

### BRIEF DESCRIPTION OF THE FIGURES

FIG. 1 depicts the change in fulvestrant plasma concentration over time upon administration of a reference composition (similar to Faslodex) or compositions of the inventive subject matter in rats.

### DETAILED DESCRIPTION OF THE INVENTION

The inventive subject matter provides ready to inject compositions with improved solubility and stability. In particular, compositions comprising fulvestrant or other hormone therapy drug at a concentration of greater than 100 mg/ml are provided, which include DEGEE-containing solvent systems, and maintain degradation of the fulvestrant at a level of less than 5 wt % when stored over at least three months at 25° C.

For example, in experiments showing the solubility and stability of compositions of the inventive subject matter as further discussed below, a ready to inject fulvestrant composition was formulated, including 300 mg fulvestrant, 4 v/v % benzyl alcohol, and DEGEE in a quantity sufficient to make up 1.7 ml. The fulvestrant solubility achieved was 176 mg/ml.

However, as shown in the following examples, it should be appreciated that high solubility and stability fulvestrant compositions do not need to be limited to formulations having solvent systems consisting of DEGEE and benzyl alcohol. Contemplated formulations can include various concentrations and combinations of DEGEE, a benzyl alcohol co-solvent, one or more other co-solvents, and one or more release rate modifiers.

### Example 1

### Solubility

Solubility studies of fulvestrant were performed using various combinations of solvent, co-solvents, oils and release rate modifiers. The resultant data are shown herein below Table 1.

TABLE 1

| | | Fulvestrant |
|---|---|---|
| Sr. | Solvent/Mixture of solvents | solubility |
| No. | and release rate modifiers | achieved |
| 1. | N-methyl Pyrolidone | 250 mg/ml |
| 2. | TCLS-101 (DMI) | 30.76 mg/ml |
| 3. | Polyethylene Glycol 400 | 11.11 mg/ml |
| 4. | Benzyl alcohol (2 v/v %):Diethylene glycol | 200 mg/mL |
| | monoethyl ether ((q.s. to 1 mL) | |
| 5. | Benzyl alcohol (2 v/v %):MCT oil (1 v/v | 200 mg/mL |
| | %):Diethylene glycol monoethyl ether ((q.s. | |
| | to 1 mL) | |
| 6. | Benzyl alcohol (4 v/v %):Diethylene glycol | 300 mg/1.7 mL |
| | monoethyl ether ((q.s. to 1.7 mL) | 176 mg/ml |
| 7. | Benzyl alcohol (5 v/v %):Diethylene glycol | 500 mg/3.3 mL |
| | monoethyl ether ((q.s. to 3.3 mL) | 151 mg/ml |
| 8. | Benzyl alcohol (4 v/v %):Diethylene glycol | 125 mg/mL |
| | monoethyl ether (46 v/v %):Castor oil (q.s. | |
| | to 1 mL) | |
| 9. | Benzyl alcohol (4 v/v %):Diethylene glycol | 30 mg/mL |
| | monoethyl ether (31 v/v %):Castor oil (q.s. | |
| | to 1 mL) | |
| 10. | Benzyl alcohol (10 w/v %):Ethanol (10 w/v | 50 mg/mL |
| | %):Benzyl benzoate (15 w/v %):Castor oil | |
| | (q.s. to 1 mL) | |
| 11. | Benzyl alcohol (10 v/v %):Castor oil (50 v/v | 151 mg/mL |
| | %):Diethylene glycol monoethyl ether (q.s. | |
| | to 1 mL) | |
| 12. | Benzyl alcohol (4 v/v %):Benzyl benzoate (10 | 56 mg/mL |
| | w/v %):Castor oil (50 v/v %):Diethylene | |
| | glycol monoethyl ether (q.s. to 1 mL) | |

As shown, various high solubility fulvestrant formulations were formulated using a solvent system comprising or consisting of DEGEE and between 2-5 v/v % benzyl alcohol. A small concentration of oil (e.g., MCT oil) as a release rate modifier and second co-solvent did not substantially affect the fulvestrant solubility. Furthermore, a high solubility fulvestrant formulation was achieved even with higher concentrations of benzyl alcohol (e.g., 10 v/v %) where a high concentration of castor oil (e.g., 50 v/v %) was present. Where one or more of benzyl alcohol, ethanol and benzyl benzoate were present in larger concentrations (e.g., 14-35 v/v %), the fulvestrant solubility achieved was lower, for example, similar to Faslodex.

### Example 2

### Method of Manufacturing Ready to Inject High Solubility Fulvestrant Composition

Fulvestrant at a concentration of 1-20 w/v % is added to minimum quantity of DEGEE and stirred. 1% Benzyl alcohol is added while stirring. The ingredients are mixed well to dissolve. The solution is diluted further q.s. with DEGEE to make up the volume to 1 ml (See Table 2). The same is filtered aseptically and filled in ampoules or vials under nitrogen bubbling and blanketing.

TABLE 2

| | | |
|---|---|---|
| Sr. No. | Name of Ingredients | Quantity per ml |

| | | | |
|---|---|---|---|
| 1. | Fulvestrant | 10-200 | mg |
| 2. | Benzyl alcohol | 1.0% | v/v |

| | | |
|---|---|---|
| 3. | Diethylene glycol monoethyl ether | Q.s. to 1 ml |

### Example 3

### Method of Manufacturing Ready to Inject High Solubility Fulvestrant Composition

Fulvestrant at a concentration of 10 w/v %/o is added to minimum quantity of DEGEE and stirred. 2% Benzyl alcohol is added while stirring. The ingredients are mixed well to dissolve. The solution is diluted further q.s. with DEGEE to make up the volume to 1 ml (See Table 3). The same is filtered aseptically and filled in ampoules or vials under nitrogen bubbling and blanketing.

TABLE 3

| | | |
|---|---|---|
| Sr. No. | Name of Ingredients | Quantity per ml |

| | | | |
|---|---|---|---|
| 1. | Fulvestrant | 100 | mg |
| 2. | Benzyl alcohol | 2.0% | v/v |

| | | |
|---|---|---|
| 3. | Diethylene glycol monoethyl ether | Q.s. to 1 ml |

### Example 4

### Method of Manufacturing Ready to Inject High Solubility Fulvestrant Composition

Fulvestrant at a concentration of 15 w/v % is added to minimum quantity of DEGEE and stirred. 4% Benzyl alcohol is added while stirring. The ingredients are mixed well to dissolve. The solution is diluted further q.s. with DEGEE to make up the volume to 1 ml (See Table 4). The same is filtered aseptically and filled in ampoules or vials under nitrogen bubbling and blanketing. The solution viscosity of this formulation was found to be about 6.124 cps.

TABLE 4

| | | |
|---|---|---|
| Sr. No. | Name of Ingredients | Quantity per ml |

| | | | |
|---|---|---|---|
| 1. | Fulvestrant | 150.0 | mg |
| 2. | Benzyl alcohol | 4.0% | v/v |

| | | |
|---|---|---|
| 3. | Diethylene glycol monoethyl ether | Q.s. to 1 ml |

### Example 5

### Impurities

A fulvestrant composition was prepared as taught herein in Example 4 (150 mg fulvestrant, 4 v/v % benzyl alcohol, and DEGEE q.s. to 1 ml), and was filtered aseptically and filled in ampoules or vials under nitrogen bubbling and blanketing. The composition was tested for 6 months stability studies to assess drug degradation patterns. The impurities levels were calculated using area normalization method (USP 39). 6 months total impurities results was found to be encouraging as 0.25% (at 2-8° C.), 0.25% (at 25° C./60% R.H.) and 0.17% (at 30° C./65% R.H.), respectively, which are each less than 0.5 wt %.

### Example 6

### Effect of Oxygen Content on Stability of Fulvestrant Injection Compositions

Upon achieving success in the initial experiments set forth above, further trials were taken to establish the stability of the formulation of Example 4 for up to 180 days. The fulvestrant composition was prepared and filtered aseptically and filled in vials under nitrogen bubbling and blanketing. The vials were exposed to various percentages of oxygen to determine the degradation of the composition. The composition was tested for Related Compounds as per USP 39 API method (by Area normalization method in HPLC). The following experiments were performed to determine the impact of ambient oxygen content (in the head space of vials) on the stability of inventive formulations with respect to the content of impurities:
1. Effect of approximately 10% oxygen content (11.50% by volume according to gas chromatography testing) in head space of filled vials.
2. Effect of approximately 15% oxygen content (16.53% by volume according to gas chromatography testing) in head space of filled vials; and
3. Effect of approximately 20% oxygen content (20.70% by volume according to gas chromatography testing) in head space of filled vials.

The results obtained are presented in Tables 5-7 below. * OC: Oxygen content; **ND: Not detected. The analytical variations in 30 and 180 days stability data could be attributed to adopted Area normalization method. The results below clearly indicate that the composition is physically and chemically stable for up to 180 days at all ICH conditions in the presence of a high oxygen concentration environment.

TABLE 5

| Sr. | | Test | | 2-8° C. | |
|---|---|---|---|---|---|
| No. | Formulation | Parameters | Initial | 30 Days | 180 Days |
| 1 | Formulation | Description | Clear | Clear | Clear |
| | of Example | | colorless | colorless | colorless |
| | 4 (with | | liquid | liquid | liquid |
| | 11.50% | Total | 0.06% | 0.118% | ND** |
| | OC*) | Impurities | | | |
| | | (NMT 1.0%) | | | |
| | | Assay | 100.69% | 101.80% | 100.50% |
| 2 | Formulation | Description | Clear | Clear | Clear |
| | of Example | | colorless | colorless | colorless |
| | 4 (with | | liquid | liquid | liquid |
| | 16.53% | Total | 0.06% | 0.101% | ND** |
| | OC*) | Impurities | | | |
| | | (NMT 1.0%) | | | |
| | | Assay | 101.31% | 102.11% | 98.72% |
| 3 | Formulation | Description | Clear | Clear | Clear |
| | of Example | | colorless | colorless | colorless |
| | 4 (with | | liquid | liquid | liquid |
| | 20.70% | Total | 0.06% | 0.087% | 0.088% |
| | OC*) | Impurities | | | |
| | | (NMT 1.0%) | | | |
| | | Assay | 104.20% | 102.36% | 99.99% |

TABLE 6

| Sr. | | Test | | 25° C./60% R.H. | |
|---|---|---|---|---|---|
| No. | Formulation | Parameters | Initial | 30 Days | 180 Days |
| 1 | Formulation | Description | Clear | Clear | Clear |
| | of Example | | colorless | colorless | colorless |
| | 4 (with | | liquid | liquid | liquid |
| | 11.50% | Total | 0.06% | 0.106% | ND** |
| | OC*) | Impurities | | | |
| | | (NMT 1.0%) | | | |
| | | Assay | 100.69% | 100.21% | 99.79% |
| 2 | Formulation | Description | Clear | Clear | Clear |
| | of Example | | colorless | colorless | colorless |
| | 4 (with | | liquid | liquid | liquid |
| | 16.53% | Total | 0.06% | 0.091% | ND** |
| | OC*) | Impurities | | | |
| | | (NMT 1.0%) | | | |
| | | Assay | 101.31% | 101.82% | 97.88% |
| 3 | Formulation | Description | Clear | Clear | Clear |
| | of Example | | colorless | colorless | colorless |
| | 4 (with | | liquid | liquid | liquid |
| | 20.70% | Total | 0.06% | 0.108% | ND** |
| | OC*) | Impurities | | | |
| | | (NMT 1.0%) | | | |
| | | Assay | 104.20% | 101.42% | 100.10% |

TABLE 7

| Sr. | | Test | | 40° C./75% R.H. | |
|---|---|---|---|---|---|
| No. | Formulation | Parameters | Initial | 30 Days | 180 Days |
| 1 | Formulation | Description | Clear | Clear | Clear |
| | of Example | | colorless | colorless | colorless |
| | 4 (with | | liquid | liquid | liquid |
| | 11.50% | Total | 0.06% | 0.124% | ND** |
| | OC*) | Impurities | | | |
| | | (NMT 1.0%) | | | |
| | | Assay | 100.69% | 100.99% | 98.53% |
| 2 | Formulation | Description | Clear | Clear | Clear |
| | of Example | | colorless | colorless | colorless |
| | 4 (with | | liquid | liquid | liquid |
| | 16.53% | Total | 0.06% | 0.130% | ND** |
| | OC*) | Impurities | | | |
| | | (NMT 1.0%) | | | |
| | | Assay | 101.31% | 100.81% | 101.27% |
| 3 | Formulation | Description | Clear | Clear | Clear |
| | of Example | | colorless | colorless | colorless |
| | 4 (with | | liquid | liquid | liquid |
| | 20.70% | Total | 0.06% | 0.151% | ND** |
| | OC*) | Impurities | | | |
| | | (NMT 1.0%) | | | |
| | | Assay | 104.20% | 102.27% | 101.27% |

### Example 7-12

### Fulvestrant Compositions with Benzyl Benzoate or Castor Oil

Fulvestrant compositions can be prepared based on the teachings herein, which include other excipients in variable concentration as shown below in Table 8. All of the following formulations were clear and physically stable when preserved in cold and at room temperature for a period of 15 days.

TABLE 8

| Sr. | | Quantity per ml | | | | | |
|---|---|---|---|---|---|---|---|
| No. | Ingredients | 7 | 8 | 9 | 10 | 11 | 12 |
| 1. | Fulvestrant | 150 mg | 150 mg | 150 mg | 150 mg | 111.11 mg | 125.0 mg |
| 2. | Benzyl benzoate | 50% w/v | 50% w/v | 20% w/v | 20% w/v | — | — |
| 3. | Benzyl alcohol | — | 4% w/v | — | 4% w/v | 4% w/v | 10% w/v |
| 4. | Diethylene | q.s. to | q.s. to | q.s. to | q.s. to | 36% w/v | 40% w/v |
| | glycol monoethyl | 1 ml | 1 ml | 1 ml | 1 ml | | |
| | ether | | | | | | |
| 5. | Castor oil | — | — | — | — | q.s. to | q.s. to |
| | | | | | | 1 ml | 1 ml |

### Example 13

### Pharmacokinetic Study

A single dose comparative pharmacokinetic study of two test formulations prepared according to the present inventive subject matter was performed against a reference formulation similar to Faslodex (prepared based on patent publication WO 2001051056 A1 to Astrazeneca), in a female Sprague Dawley Rat model. See Table 9 for reference and test formulations.

TABLE 9

| | | Quantity/ml | | | | | |
|---|---|---|---|---|---|---|---|
| | | Batch No.: | | Batch No.: | | Batch No.: | |
| | | FLV-05 | | FLV-11 | | FLV-06 | |
| | | (Test Formu- | | (Test Formu- | | (Reference | |
| S. | | lation-1) | | lation-2) | | Formulation) | |
| No. | Ingredients | 500 mg/3.5 ml | | 500 mg/5 ml | | 250 mg/5 ml) | |
| 1 | Fulvestrant USP | 150.0 | mg | 100.0 | mg | 50.0 | mg |
| 2 | Benzyl alcohol | 4.0% | v/v | 10.0% | v/v | 10.0% | w/v |
| | BP | | | | | | |
| 3 | Diethylene glycol | Q.s. to 1 ml | | Q.s. to 1 ml | | — | |
| | monoethyl ether | | | | | | |
| | (Transcutol HP) | | | | | | |
| 4 | Ethanol BP | — | | — | | 10.0% | w/v |
| 5 | Benzyl benzoate | — | | — | | 15.0% | w/v |
| 6 | Castor oil BP | — | | 45.0% | v/v | Q.s. to 1 ml | |
| | Viscosity | 6.124 | cps | 40.778 | cps | 86.470 | cps |

18 healthy rats were distributed into three different groups (6 rats each). The reference formulation was intramuscularly administered to a first group, a first test formulation was intramuscularly administered to a second group, and a second test formulation was intramuscularly administered to a third group. Each formulation was administered at a dose of 10 mg/kg body weight. Blood samples were collected from the retro orbital plexus over a 30 day period following dosing to analyze pharmacokinetic parameters. The blood samples were collected on days 1, 2, 3, 4, 5, 6, 7, 10, 15, 20 and 30 post-dosing for the analysis.

The mean log transformed Cₘₐₓ, AUC₀₋ₜ and AUC_{0-∞} data observed during the study of the test and reference formulations are summarized in below Table 10.

TABLE 10

| | Parameters | | |
|---|---|---|---|
| | Cₘₐₓ | AUC₀₋ₜ | AUC_{0-∞} |
| | (ng/mL) | (ng · hr/mL) | (ng · hr/mL) |
| Reference | 18.0396 | 5986.0880 | 8986.3031 |
| Formulation | | | |
| Test Formulation 1 | 23.2409 | 6907.3583 | 8797.8799 |
| Test Formulation 2 | 17.5162 | 4608.9614 | 8094.2816 |

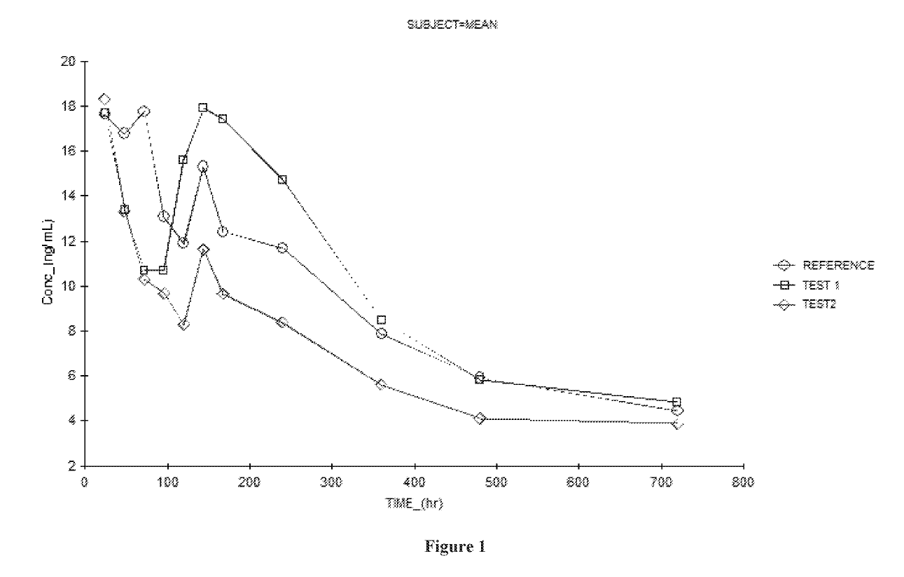

The above results indicate that Cₘₐₓ (the maximum concentration available in the blood) of test formulation 1 is slightly higher than the reference product. The Cₘₐₓ of test formulation 2 is similar to the Cₘₐₓ of the reference product. The AUC₀₋ₜ and the AUC_{0-∞} results show that the maximum concentration of the drug is effectively available in plasma using the test formulations, and indicates that a better extent and rate of absorption of the drug can be achieved than that of the currently marketed Faslodex. FIG. 1 depicts fulvestrant plasma concentrations over time upon administration of each of the formulations to the rats. Each data point represents the mean plasma fulvestrant concentration of a group (n=6 rats per group). The results of this experiment show that the test formulations are safe, and do not appear to exceed the toxicity level of the reference product. This indicates that the test formulations can allow for rapid penetration and enhanced absorption as compared to the simultaneously prepared reference product when administered intramuscularly.

The two test formulations not only showed improved fulvestrant solubility (and higher mg/ml concentrations), but also showed significantly lower visocities and improved syringeability when compared to the reference formulation. Such formulations can advantageously reduce the pain and burden felt by the patient receiving the injection, while reducing the difficulties for healthcare professionals administering the drug by reducing the time and force required to deliver a suitable dose.

Therefore, various improved high solubility and stability fulvestrant compositions that can be administered in smaller volumes with reduced pain are provided, as well as methods for preparing such compositions, and methods for using such compositions to treat or prevent a disease or disorder.

It should be appreciated that it is an object of the inventive subject matter to provide a stable, physiologically effective composition comprising fulvestrant or other hormone therapy drug, alone or in combination with other pharmaceutically effective ingredients or drugs, which is suitable to be administered parenterally, particularly via intramuscular injection. It is also an object of the inventive subject matter to provide fulvestrant compositions with improved bioavailability and reduced toxicity (relative to known fulvestrant compositions), which are easily syringeable and administrable. It is yet another object of the invention to provide therapeutically effective amounts of fulvestrant in a fulvestrant composition that can be intramuscularly injected in smaller volumes and with reduced pain.

The optimum therapeutically effective amount of a drug is the amount of the drug in the composition that will yield the most effective results in terms of efficacy of treatment in a given subject. This amount can vary depending upon a variety of factors, including but not limited to the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

The formulations of the inventive subject matter can be administered according to any suitable dosing schedule. For example, it is contemplated that a dose of between 100-1,000 mg fulvestrant, more preferably between 150-750 mg fulvestrant, and even more preferably between 200-550 mg fulvestrant can be administered once, twice, or even three or more times per month.

Although some preferred compositions according to the inventive subject matter may be administered via intramuscular injection, it is contemplated that the formulations can be used to form a dosage form administered in any suitable manner, including for example, orally via capsules, powders, tablets, troches, elixirs, suspensions, syrups, wafers, chewing gums, aqueous suspensions or solutions. Oral pharmaceutical preparations can be made following the conventional techniques of pharmacy involving milling, mixing, granulation, and compressing, when necessary, for tablet forms; or milling, mixing and filling for hard gelatin capsule forms. When the dosage unit form is a capsule, it may additionally contain a pharmaceutically acceptable carrier, such as a liquid carrier (e.g., a fatty oil).

Other dosage unit forms may contain other various materials which modify the physical form of the dosage unit, such as, for example, a coating. Thus, tablets or pills may be coated with sugar, shellac, or other enteric coating agents. Materials used in preparing these various compositions should be pharmaceutically or veterinarally pure and non-toxic in the amounts used. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation.

Other suitable routes of administration may include parenteral, inhalation, topical, rectal, nasal, or via an implanted reservoir or trans-dermal patch, wherein the term “parenteral” as used herein includes subcutaneous, intravenous, intramuscular, intraarticular, intrasynovial, intrathecal, intrahepatic, intralesional, and intracranial administration (typically injection or infusion).

Furthermore, the liquid compositions presented herein can have a viscosity such that they can be filled into a pump spray as a spray formulation or into a vaporizer such as nebulizer for use in oral or nasal administration. For example, the compositions prepared as described herein can have a viscosity from of between 1-45 centipoise (cps), or between 1-7 cps at room temperature.

The administration of the suitable dose can be administered with a single administration, or can be spread out over the course of a day through multiple administrations. For example, an effective dose of the composition can be divided and separately packaged in a pre-filled syringe or vial, or in a set of syringes or vials (e.g., 2, 3, 4, 5 syringes or vials). Additionally or alternatively, the suitable dose can be divided and separately packaged in one or more capsules, tablets, powders or oral dissolve strips, and separately administered one to five or more times a day. Alternate day dosing or dosing once every several days may also be utilized.

Contemplated formulations may also include one or more anti-oxidants. For example, hydrophobic anti-oxidants include butylated hydroxytoluene, butylated hydroxyanisole, propyl gallate, and α-tocopherol, DL-tocopherol, α-tocopherol acetate, Tocopherol Polyethylene Glycol Succinate (Vitamin E TPGS), L-cysteine, or hydrophilic anti-oxidants, including sodium EDTA and thioglycerol. Most typically, the concentration of the anti-oxidant can be between 0.005% and 10% w/v of the total composition. Additionally, or alternatively, contemplated formulations may include a preservative (e.g., phenol, thimerosal, chlorobutanol, m-cresol, phenoxyethanol, methylparaben and propylparaben), typically at a concentration of between 0.001% w/v and less than 10% w/v of the total composition. For example, contemplated compositions can include ethanol at 1-4 w/v % (although some preferred compositions are free or essentially free of ethanol), chlorobutanol at 0.1-2 w/v %, parabens such as methyl paraben 0.1-0.18 w/v % or propyl paraben 0.01-0.2 w/v %, isosorbide dimethyl ether, glycerol, thioglycerol, phenol at 0.1-1 w/v %, meta cresol or chlorocresol at 0.1-0.3%, methylhydroxy benzoate 0.1-0.2 w/v %, or a phenyl mercuric salt such as acetate, borate or nitrate 0.1-0.2 w/v %.

The carrier may also contain adjuvants such as preserving stabilizing, wetting, emulsifying agents and the like together with the penetration enhancer. In some embodiments, the fulvestrant composition can include additional excipients e.g. preservatives for multi-dose containers, including for example, phenol, phenoxyethanol, methylparabens and propylparabens.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. The ready to inject formulations should also be able to pass readily through an injection device such as a hollow needle.

It should further be appreciated that contemplated formulations can be sterilized and all known manners of sterilization are deemed suitable for use herein, including filtration through 0.22 micron filters, heat sterilization, radiation (e.g., gamma, electron beam, microwave), or ethylene oxide sterilization to render the formulations sterile. Where contemplated formulations are lyophilized, they may be prepared as lyophilized cake, lyophilized powder, etc.

Depending on the particular purpose, it should also be recognized that contemplated compositions may be combined (in vivo, or in a therapeutic formulation or administration regimen) with at least one other therapeutically active agent to additively or synergistically provide a therapeutic or prophylactic effect. The additional ingredients could include, for example, other anticancer agents such as palbociclib or letrozole in suitable dosage form to achieve therapeutically effective blood concentration for the treatment of breast cancer.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The discussion herein provides example fulvestrant compositions and methods of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described.

Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A ready to inject fulvestrant composition, comprising:
fulvestrant present in the composition at a concentration of greater than 100 mg/ml;
diethylene glycol monoethyl ether or an alkyl derivative thereof as a solvent;
between 1-5 v/v % of a co-solvent; and
wherein the composition maintains a degradation of fulvestrant at a level of less than 5 wt % when stored over at least three months at 25° C.

2. The composition of claim 1, wherein the co-solvent is benzyl alcohol.

3. The composition of claim 1, wherein fulvestrant is present at a concentration of at least 125 mg/ml.

4. The composition of claim 3, wherein fulvestrant is present at a concentration of at least 150 mg/ml.

5. The composition of claim 1, further comprising a second co-solvent present in the composition at no more than 50 v/v %.

6. The composition of claim 5, wherein the second co-solvent comprises castor oil and acts as a release rate modifier.

7. The composition of claim 1, wherein the composition is formulated to maintain degradation of fulvestrant at a level of less than 2 wt % when stored over at least three months at 25° C.

8. The composition of claim 1, wherein the composition is formulated to maintain degradation of fulvestrant at a level of less than 5 wt % when stored over at least three months at 2-8° C.

9. The composition of claim 1, wherein the composition is provided in a multi-use container.

10. The composition of claim 1, wherein the diethylene glycol monoethyl ether or the alkyl derivative thereof is present in the composition at more than 40 v/v %.

11. A method of manufacturing a ready to inject fulvestrant composition containing article, comprising:
formulating a liquid composition including fulvestrant such that the formulation exhibits less than 5 wt % degradation of the fulvestrant when stored over at least three months at 25° C.;
wherein the liquid composition comprises diethylene glycol monoethyl ether or an alkyl derivative thereof as a solvent, fulvestrant at a concentration greater than 100 mg/ml, and between 1-5 v/v % of a co-solvent; and
packaging the liquid formulation in the article.

12. The method of claim 11, wherein the co-solvent is benzyl alcohol.

13. The method of claim 11, wherein fulvestrant is present at a concentration of at least 125 mg/ml.

14. The method of claim 13, wherein fulvestrant is present at a concentration of at least 150 mg/ml.

15. The method of claim 11, wherein the liquid composition further comprises a second co-solvent present in the composition at no more than 50 v/v %.

16. The method of claim 15, wherein the second co-solvent comprises castor oil and acts as a release rate modifier.

17. The method of claim 11, wherein formulating comprises formulating the liquid composition such that the formulation exhibits less than 2 wt % degradation when stored over at least three months at 25° C.

18. The method of claim 11, wherein formulating comprises formulating the liquid composition such that the formulation remains clear and colorless when stored over at least three months at 25° C.

19. The method of claim 11, wherein the article is a multi-use container, and wherein packaging comprises packaging the liquid formulation in an amount that includes at least 500 mg fulvestrant.

20. The method of claim 11, wherein diethylene glycol monoethyl ether or the alkyl derivative thereof is present in the composition at more than 40 v/v %.

21. A ready to inject fulvestrant composition, comprising:
fulvestrant present in the composition at a concentration of greater than 100 mg/ml;
diethylene glycol monoethyl ether or an alkyl derivative thereof as a solvent;
wherein the composition has a viscosity of less than 80 centipoise; and
wherein the composition maintains a degradation of the fulvestrant at a level of less than 5 wt % when stored over at least three months at 25° C.

22. The composition of claim 21, further comprising between 1-5 v/v % of a co-solvent.

23. The composition of claim 21, wherein the co-solvent is benzyl alcohol.
